(19) United States
(12) Patent Application Publication
(10) Pub. No.: US 2011/0142834 A1
(43) Pub. Date: Jun. 16, 2011

(54) TREATMENT OF HEARING AND BALANCE IMPAIRMENTS USING COMPOUNDS HAVING ERYTHROPOIETIN ACTIVITY

(75) Inventor: Guy M. Miller, Monte Sereno, CA (US)

(73) Assignee: Edison Pharmaceuticals, Inc.

(21) Appl. No.: 12/991,909

(22) PCT Filed: May 13, 2009
(86) PCT No.: PCT/US09/43786

§ 371 Date: Feb. 8, 2011

Related U.S. Application Data
(60) Provisional application No. 61/127,877, filed on May 15, 2008.

(51) Int. Cl. A61K 39/395; A61K 38/18; A61P 27/16; A61P 25/00

(52) U.S. Cl. 424/134.1; 514/7.7

(57) ABSTRACT

Compositions and methods are provided for prophylactic or therapeutic treatment of a mammal for hearing or balance impairments involving neuronal damage, loss, or degeneration, preferably of spiral ganglion neurons, by administration of a therapeutically effective amount of one or more molecules having erythropoietin activity selected from EPO, or a biosimilar, a variant, or a mutant thereof; a protein or peptide mimetic of EPO; a small molecule mimetic of EPO and an erythropoiesis stimulating agent. Also provided are improved compositions and methods for treatments of ototoxicity requiring administration of a pharmaceutical having an ototoxic side-effect in combination with a therapeutically effective amount of a molecule having erythropoietin activity.

### CROSS-REFERENCE TO RELATED APPLICATIONS

This application claims priority benefit of U.S. Provisional Patent Application No. 61/127,877, filed May 15, 2008. The entire content of that application is hereby incorporated by reference in its entirety.

### TECHNICAL FIELD

The present invention relates to compositions and methods for prophylactic and therapeutic treatment of hearing impairments, particularly for the treatment of noise induced, age-induced and ototoxin-induced hearing impairments involving inner hair cell damage or loss, neuronal damage, loss or degeneration of neurons in a patient, or for the prevention of toxic side effects of ototoxic medications, by administration of compounds having erythropoietin activity. The present invention also relates to compositions and methods for prophylactic and therapeutic treatment of ototoxin-induced balance impairments.

### BACKGROUND

Hearing impairments are serious handicaps which affect millions of people. Hearing impairments can be attributed to a wide variety of causes, including infections, mechanical injury, loud sounds, aging, and chemical-induced ototoxicity that damages hair cells of the peripheral auditory system and/or the primary afferent neurons in the spiral ganglia that transduce auditory signals from the hair cells to the brain.

The peripheral auditory system consists of auditory receptors, hair cells in the organ of Corti, and primary auditory neurons, the spiral ganglion neurons in the cochlea. Spiral ganglion neurons (“SGN”) are primary afferent auditory neurons that deliver signals from the peripheral auditory receptors, the hair cells in the organ of Corti, to the brain through the cochlear nerve. The eighth nerve connects the primary auditory neurons in the spiral ganglia to the brain stem. The eighth nerve also connects vestibular ganglion neurons (“VGN”), which are primary afferent sensory neurons responsible for balance and which deliver signals from the utricle, saccule and ampullae of the inner ear to the brain.

Noise induced hearing loss (NIHL) can arise under either acute or chronic circumstances. Noise induced hearing loss can give rise to multifarious problems. In addition to the inability to hear certain sounds, especially in the upper registers, individuals experiencing such hearing loss may also experience tinnitus or ringing of the ears. Additionally noise can mechanically irritate the inner ear, giving rise to an inflammatory response characterized by fluid buildup and dampening of sound transmission within the ear. Moreover, excessive noise can also give rise to a neuronal type of hearing loss. In the earlier stages of neuronal hearing loss, the patient experiences a degradation of his ability to discriminate between certain words or to understand certain persons with voices in the upper or lower registers. It has been reported that certain antioxidants, particularly idebenone and Vitamin E therapy offer a potential approach to attenuate noise induced hearing loss (Fetoni, Neuroreport (2008) Vol 19, No. 3, 277-281). Similarly Trolox has been reported to attenuate noise-induced hearing loss (Yamashita, Neuroscience (2005), 134:633-643).

Another type of hearing loss is drug-induced or chemically-induced hearing loss (CIHL). Both the vestibular and auditory systems are sensitive to ototoxic drugs, which are detrimental to hearing or balance, or both. Ototoxic drugs include therapeutic drugs, antineoplastic agents, contaminants in foods or medicaments, and environmental and industrial pollutants. Ototoxic drugs include the widely used chemotherapeutic agent cisplatin and its analogs (Fleischman, Toxicol Appl. Pharmacol. (1975) 33:320-332; Stadnicki, Cancer Chemother. Rep. (1975) 59:467-480; Nakai, Acta Otarolyngol. (1982) 93:227-232; Berggren, Acta Otarolyngol. (1990) 109; 57-65; Dublin, Fundamentals of sensorineural auditory pathology. Springfield, Ill.: C. C. Thomas (1976); Hood and Berlin, Contemporary applications of neurobiology in human hearing assessment (Raven Press, N.Y., 1986)), commonly used aminoglycoside antibiotics, e.g. gentamicin, for the treatment of infections caused by Gram-negative bacteria, (Sera et al., Scanning Microsc. (1987) 1 1191:1197; Hinojosa and Lerner, J. Infect. Dis. (1987) 156: 449-455; Bareggi et al., Pharmacol. Res. (1990) 2:635-644), quinine and its analogs, salicylate and its analogs, NSAIDs, and diuretics.

Aminoglycoside antibiotics are vital for the treatment of serious bacterial infections. However, in some patients, the antibiotics have severe toxic effects, particularly on the auditory system. The toxic effects of these drugs on auditory cells and spiral ganglion neurons are often the limiting factor for their therapeutic usefulness. For example, antibacterial aminoglycosides such as gentamicins, streptomycins, kanamycins, tobramycins, and the like are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980) or most recent edition). Aminoglycoside antibiotics are generally utilized as broad spectrum antimicrobials effective against, for example, gram-positive, gram-negative and acid-fast bacteria. Susceptible microorganisms include Escherichia spp., Hemophilus spp., Listeria spp., Pseudomonas spp., Nocardia spp., Yersinia spp., Klebsiella spp., Enterobacter spp., Salmonella spp., Staphylococcus spp., Streptococcus spp., Mycobacteria spp., Shigella spp., and Serratia spp.

As implied by the generic name for the family, all the aminoglycoside antibiotics contain aminosugars in glycosidic linkage. Ototoxicity is a dose-limiting side-effect of antibiotic administration. For example, nearly 75% of patients given 2 grams of streptomycin daily for 60 to 120 days displayed some vestibular impairment, whereas at 1 gram per day, the incidence decreased to 25% (U.S. Pat. No. 5,059,591). Auditory impairment was observed: from 4 to 15% of patients receiving 1 gram per day for greater than 1 week develop measurable hearing loss, which slowly becomes worse and can lead to complete permanent deafness if treatment continues. Ototoxicity is also a serious dose-limiting side-effect for cisplatin, a platinum coordination complex that has proven effective on a variety of human cancers including testicular, ovarian, bladder, and head and neck cancer. Cisplatin damages auditory and vestibular systems (Toxicol. Appl. Pharmacol. (1975) 33:320-332; Stadnicki, Cancer Chemother. Rep. (1975) 59:467-480; Nakai, Acta Otarolyngol. (1982) 93:227-232; Carenza, Gynecol. Oncol., (1986) 25:244-249; Sera, Scanning Microsc. (1987) 1 1191:1197; Bareggi, et al., Pharmacol. Res. (1990) 2:635-644)). The effect of Erythropoietin on Gentamicin induced auditory hair cell loss has been described by Monge, A. et al, The Laryngoscope, (2006), 116:312-316. Hahn, H, et al, (see Abstract Book, 42^{nd} Workshop on Inner Ear Biology, Tuebingen, Germany (2005) p. 48) analyzed the EPO/EPO-receptor system under the conditions of acoustic trauma and discussed EPO as a putative protective substance of the inner ear.

Over the last few years hearing loss has been found to be associated with a multitude of different mitochondrial defects. Patients with generalized neuromuscular dysfunction also manifest hearing deficits. Description of several families with mitochondrial mutations, in whom sensorineural hearing loss and diabetes mellitus occur with significant penetrance but not always together are known (Ballinger et al., Nature Genetics 1, (1992) 11-15; van den Ouweland et al., Nature Genet 1; (1992) 368-371; Reardon et al, Lancet 340, (1992) 1376-1379). Even more surprisingly, in most of these families the pathogenic heteroplasmic mutation is the same A→G transition mutation at nucleotide 3243 in the mitochondrial gene for tRNAleu(UUR) as in MELAS (van den Ouweland et al, 1992; Reardon et al, 1992). This association between diabetes mellitus, hearing loss, and mitochondrial mutations has been confirmed in population studies of diabetic patients (Oka et al., Lancet 342, (1993)527-528; Kadowaki et al., New Engl J Med 330, (1994) 962-968; Alcolado et al., Diabetologia 37, (1994) 372-376; Katagiri et al., Diabetologia 37, (1994) 504-510). Kadowaki et al., for example, found the heteroplasmic nucleotide 3243 mutation in 2-6% of diabetic patients in Japan, and in 3 out of 5 patients with diabetes and deafness. 27 of their 44 patients with diabetes and the nucleotide 3243 mutation also had hearing loss. The hearing loss is sensorineural, and usually develops only after the onset of diabetes.

There exists a need for means to prevent, reduce or treat the incidence and/or severity of hearing impairments involving auditory nerves, particularly that arising as an unwanted side-effect of ototoxic therapeutic drugs including cisplatin and its analogs, aminoglycoside antibiotics including gentamicin and analogs, salicylate and its analogs, and loop diuretics. In addition, there exists a need for methods which will allow higher and thus more effective dosing with these ototoxicity-inducing pharmaceutical drugs, while concomitantly preventing or reducing ototoxic effects caused by these drugs. What is needed is a method that provides a safe, effective, and prolonged means for prophylactic or curative treatment of hearing impairments related to nerve damage, loss, or degeneration, particularly ototoxin-induced. In addition there is needed a rapid, reliable, and facile system for testing the effects and mechanisms of ototoxic agents on hearing in animals, including humans, and for testing the efficacy of therapeutics to prevent, reduce or treat these impairments. The present invention provides a method and system to achieve these goals and others as well.

### DISCLOSURE OF THE INVENTION

The present invention is based on the discovery disclosed herein that administration of certain compositions comprising compounds having erythropoietin activity can prevent or reduce hearing impairments. The hearing impairments are due to inner ear hair cell damage or loss, or neuronal damage, wherein the damage or loss is caused by infection, mechanical injury, aging, noise, acoustic trauma, or chemical-induced ototoxicity. The compounds of the present invention may be administered to promote the protection, survival, or regeneration of hair cells and spiral ganglion neurons, thus reversing, enhancing, reducing, or preventing hearing loss. Damage to the peripheral auditory system is responsible for a majority of balance deficits (Dublin, Fundamentals of Sensorineural Auditory Pathology (Chapter 3), Springfield, Ill.: Charles C. Thomas 18-103 (1976); Lim, Am. J. Otolaryngol. 7(2):73-99 (1986)) with destruction of vestibular ganglia neurons as a major cause of balance impairments. The present invention also addresses the treatment of balance impairments caused by infections, mechanical injury, loud sounds, aging, and chemical-induced ototoxicity that damage neurons and/or hair cells of the peripheral vestibular systems of the inner ear.

In one embodiment, the invention relates to a method for treating a patient having or prone to having a hearing impairment with a prophylactically or therapeutically effective amount of a composition comprising one or more molecules having erythropoietin (EPO) activity, to prevent, reduce, or treat the incidence of or severity of the hearing impairment.

In another embodiment, the invention relates to a method of reversing hearing loss, or recovering or enhancing hearing function with a prophylactically or therapeutically effective amount of a composition comprising one or more molecules having erythropoietin (EPO) activity. In some embodiments, the composition comprising one or more molecules having EPO activity can be EPO or a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a composition comprising a therapeutic amount of a pharmaceutical drug that can cause hearing loss selected from the group consisting of an aminoglycoside antibiotic, a chemotherapeutic agent, a salicylate or salicylate-like compound, a non-steroidal anti-inflammatory drug, a diuretic, a narcotic analgesic, and a quinine, and a molecule having erythropoietin activity, wherein the composition has reduced or no ototoxic effects when administered to a patient in need of a treatment with said pharmaceutical drug, with the proviso that if the aminoglycoside antibiotic is gentamicin, the molecule having erythropoietin activity is not EPO.

In one embodiment, the invention relates to a method for treating a patient having or prone to having a noise-induced hearing impairment or an acoustic trauma, to prevent, reduce, or treat the incidence of or severity of the hearing impairment with a prophylactically or therapeutically effective amount of one or more molecules having EPO activity, wherein said EPO can be an EPO biosimilar, an EPO variant, or an EPO mutant; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method for treating a patient having or prone to having an age-induced hearing impairment with a prophylactically or therapeutically effective amount of one or more molecules having EPO activity, wherein said EPO can be EPO or a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method for treating a patient with an ototoxin-induced or -inducible hearing impairment, to prevent, reduce, or treat the incidence of or severity of the hearing impairment with a prophylactically or therapeutically effective amount of a prophylactically or therapeutically effective amount of one or more molecules having EPO activity, wherein said EPO can be EPO or a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). In one embodiment, the one or more molecules having EPO activity excludes EPO itself. In another embodiment, the ototoxin excludes gentamicin.

In another embodiment, the invention relates to a method of preventing or treating ototoxicity in a patient undergoing treatment with a pharmaceutical drug having an ototoxic-hearing impairment side effect, with a therapeutically effective amount of one or more molecules having EPO activity to prevent or treat the ototoxicity induced by the pharmaceuticals. In another embodiment, the invention relates to a method of treating a patient undergoing an antibiotic, an antimicrobial, or an antifungal treatment with a pharmaceutical having an ototoxic-hearing impairment side effect, with a therapeutically effective amount of a one or more molecules having EPO activity to treat the ototoxicity induced by said antibiotics or antimicrobials. In another embodiment, the invention relates to a method of treating a patient undergoing a treatment with an aminoglycoside antibiotic, having an ototoxic-hearing impairment side effect, with a therapeutically effective amount of a one or more molecules having EPO activity to treat the ototoxicity induced by said aminoglycosides. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating a patient undergoing a treatment with an aminoglycoside antibiotic, not gentamicin, having an ototoxic-hearing impairment side effect, with a therapeutically effective amount of a one or more molecules having EPO activity such as EPO, an EPO biosimilar, an EPO variant, or an EPO mutant; a protein or peptide mimetic of EPO; a small molecule mimetic of EPO or an erythropoiesis stimulating agent (ESA), to treat the ototoxicity induced by said aminoglycoside. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). Examples of such aminoglycoside antibiotics include but are not limited to streptomycins, kanamycins, tobramycins, and the like.

In another embodiment, the invention relates to a method of treating a patient undergoing a treatment with gentamicin, having an ototoxic-hearing impairment side effect, with a therapeutically effective amount of one or more molecules having EPO activity such as an EPO biosimilar, an EPO variant, or an EPO mutant; a protein or peptide mimetic of EPO; or a small molecule mimetic of EPO, to treat the ototoxicity induced by said aminoglycoside. In another embodiment, the invention relates to a method of treating a patient undergoing a treatment with gentamicin, having an ototoxic-hearing impairment side effect, with a therapeutically effective amount of an EPO-mimetic antibody fusion protein such as CNTO-528 or CNTO-530. In other embodiments, the invention relates to a method of treating a patient undergoing a treatment with gentamicin, having an ototoxic-hearing impairment side effect, with a therapeutically effective amount of Dynepo (Epoetin delta) or CEPO (carbamylated EPO).

In another embodiment, the patient in need of a hearing impairment treatment is undergoing a treatment with an aminoglycoside antibiotic such as neomycin, amikacin, tobramycin, viomycin, sisomicin, netimicin, streptomycin, dibexacin, fortimcin, minocyclin, erythromycin, and dihydrostreptomycin with EPO.

In another embodiment, the invention relates to a method of treating a patient having a neurotoxin induced hearing impairment with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). Examples of neurotoxins are glutamates and aspartates, such as glutamic acid, glutamate, aspartic acid, aspartate, and salts and esters thereof.

In another embodiment, the invention relates to a method of treating a patient with hearing impairments resulting from the administration of quinine or its synthetic substitutes with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating a patient with hearing impairments resulting from the administration of diuretics, for example bendroflumethiazide, bumetanide, chlor-thalidone, furosemide, ethacrynic acid and mercurials, with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating a patient with hearing impairments resulting from the administration of anti-neoplastics, such as platinum-containing antineoplastic agents, with a therapeutically effective amount of a one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). Examples of anti-neoplastic drugs are cisplatin or cisplatin-like compounds, methotrexate, taxol, carboplatinum, nitrogen mustard, vinblastin, vincristine, or bleomycine.

In another embodiment, the invention relates to a method of treating a patient with hearing impairments resulting from the administration of salicylate or NSAIDs, i.e. aspirin, salicylate-like compounds, diclofenac, naproxen, ibuprofen, etodolac, ketorolac, indomethacin, piroxicam or sulindac, with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating a patient who cannot detect small changes in tone intensity, with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating a patient who cannot continue to perceive a constant tone above the threshold of hearing, with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating damage to spiral ganglion neurons with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method of treating tinnitus with a therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In some embodiments, the individual treated for tinnitus is suffering from Meniere's disease. In other embodiments the molecule having EPO activity is a small molecule mimetic of EPO such as CNTO-528 or CNTO-530. In other embodiments, the molecule having EPO activity is Dynepo (Epoeitin delta) or carbamylated EPO (CEPO).

In another embodiment, the invention relates to a composition comprising a medicament known to have an ototoxic-hearing impairment side-effect in combination with a therapeutically effective amount of one or more molecules having EPO activity such as EPO, a biosimilar, a variant, or a mutant thereof; a protein or peptide mimetic of EPO; or a small molecule mimetic of EPO for administration to a patient in need of hearing impairment treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In some embodiments, the compositions comprise aminoglycoside antibiotics and one or more molecules having EPO activity such as a biosimilar, a variant, or a mutant of EPO; a protein or peptide mimetic of EPO; a small molecule mimetic of EPO or an erythropoiesis stimulating agent (ESA) for administration to a patient in need of hearing impairment treatment. Other examples include compositions comprising gentamicin and one or more molecules having EPO activity such as a biosimilar, a variant, or a mutant of EPO; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In some embodiments, the compositions comprise aminoglycoside antibiotics excluding gentamicin and one or more molecules having EPO activity such as EPO, a biosimilar, a variant, or a mutant thereof; a protein or peptide mimetic of EPO; a small molecule mimetic of EPO or an erythropoiesis stimulating agent (ESA) for administration to a patient in need of hearing impairment treatment. Other examples include compositions comprising gentamicin and one or more molecules having EPO activity such as a biosimilar, a variant, or a mutant of EPO; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In other embodiments, the invention relates to a composition including a combination of anti-neoplastic drugs such as cisplatin or cisplatin-like compounds and one or more molecules having EPO activity such as a biosimilar, a variant, or a mutant of EPO; a protein or peptide mimetic of EPO; a small molecule mimetic of EPO or an erythropoiesis stimulating agent (ESA) for administration to a patient in need of hearing impairment treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In other embodiments, the invention relates to a composition including a combination of anti-neoplastic drugs such as cisplatin or cisplatin-like compounds and EPO.

In other embodiments, the invention relates to a composition including a combination of a neurotoxin drug such as aspartate or glutamate and one or more molecules having EPO activity such as a biosimilar, a variant, or a mutant of EPO; a protein or peptide mimetic of EPO; a small molecule mimetic of EPO or an erythropoiesis stimulating agent (ESA) for administration to a patient in need of hearing impairment treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In other embodiments, the invention relates to a composition including a combination of a neurotoxin drug such as aspartate or glutamate and EPO.

In another embodiment, the invention relates to a composition comprising a medicament known to have an ototoxic-hearing impairment side-effect in combination with one or more molecules having EPO activity such as a biosimilar, a variant, or a mutant of EPO; a protein or peptide mimetic of EPO; a small molecule mimetic of EPO or an erythropoiesis stimulating agent (ESA) for administration to a patient in need of hearing impairment treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a composition comprising a medicament known to have an ototoxic-hearing impairment side-effect, wherein the medicament is not gentamicin, in combination with one or more molecules having EPO activity such as EPO, a biosimilar, a variant, or a mutant thereof; a protein or peptide mimetic of EPO; a small molecule mimetic of EPO or an erythropoiesis stimulating agent (ESA) for administration to a patient in need of hearing impairment treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a composition comprising a medicament known to have an ototoxic-hearing impairment side-effect in combination with an erythropoietic mimetic antibody fusion protein such as CNTO-528 or CNTO-530, said composition being for administration to a patient in need of a hearing impairment treatment.

In another embodiment, the invention relates to a composition comprising a medicament known to have an ototoxic hearing impairment side-effect in combination with an erythropoietic mimetic antibody fusion protein such as CNTO-528 or CNTO-530 and an additional antioxidant or a spin-trapping agent for administration to a patient in need of a hearing impairment treatment. Examples of antioxidants include but are not limited to allopurinol, glutathione, methionine, carnitine, and ebselen.

In another embodiment, the invention relates to a composition comprising a medicament known to have an ototoxic-hearing impairment side-effect in combination with Dynepo (Epoetin delta), said composition being for administration to a patient in need of a hearing impairment treatment.

In another embodiment, the invention relates to a composition comprising a medicament known to have an ototoxic hearing impairment side-effect in combination with Dynepo (Epoetin delta) and an additional antioxidant or a spin-trapping agent a hearing impairment. Examples of antioxidants include but are not limited to allopurinol, glutathione, methionine, carnitine, and ebselen.

In another embodiment, the invention relates to a composition comprising a medicament known to have an ototoxic-hearing impairment side-effect in combination with CEPO (carbamylated EPO), said composition being for administration to a patient in need of such treatment.

In another embodiment, the invention relates to a composition comprising a medicament known to have an ototoxic hearing impairment side-effect in combination with CEPO (carbamylated EPO) and an additional antioxidant or a spin-trapping agent. Examples of antioxidants include but are not limited to allopurinol, glutathione, methionine, carnitine, and ebselen.

In another embodiment, the invention relates to a method for treating a patient with an ototoxin-induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). In one embodiment, the one or more molecules having EPO activity excludes EPO itself. In another embodiment, the ototoxin excludes gentamicin.

In another embodiment, the invention relates to a method for treating a patient with an aminoglycoside antibiotic induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). In one embodiment, the one or more molecules having EPO activity excludes EPO itself. In another embodiment, the aminoglycoside antibiotic excludes gentamicin.

In another embodiment, the invention relates to a method for treating a patient with gentamicin induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In one embodiment, the one or more molecules having EPO activity excludes EPO itself. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method for treating a patient with an anti-neoplastic induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). In some embodiments, the anti-neoplastic drug is cisplatin or cisplatin-like compounds.

In another embodiment, the invention relates to a method for treating a patient with a loop diuretic induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a method for treating a patient with a neurotoxin induced or -inducible balance impairment, to prevent, reduce, or treat the incidence of or severity of the balance impairment with a prophylactically or therapeutically effective amount of one or more molecules having EPO activity such as EPO, a biosimilar, a variant, or a mutant thereof; a protein or peptide mimetic of EPO; a small molecule mimetic of EPO or an erythropoiesis stimulating agent (ESA). In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO). In some embodiments, the neurotoxin is aspartate or glutamate.

In another embodiment, the invention relates to a composition comprising a medicament known to have an ototoxic-balance impairment side-effect in combination with CEPO (carbamylated EPO), said composition being for administration to a patient in need of such treatment.

In another embodiment, the invention relates to a composition comprising a medicament known to have an ototoxic-balance impairment side-effect in combination with CEPO (carbamylated EPO) and an additional antioxidant or a spin-trapping agent. Examples of antioxidants include but are not limited to allopurinol, glutathione, methionine, carnitine, and ebselen.

In another embodiment, the invention relates to a composition comprising a medicament known to have a tinnitus impairment side-effect in combination with a molecule having EPO activity selected from selected from EPO, or a biosimilar, a variant, or a mutant thereof; a protein or peptide mimetic of EPO; a small molecule mimetic of EPO and an erythropoiesis stimulating agent (ESA), said composition being for administration to a patient in need of such treatment. In other embodiments, one of the molecules having EPO activity is an EPO-mimetic antibody fusion protein such as CNTO-528 or CNTO-530. In other embodiments, one of the molecules having EPO activity is Dynepo (Epoetin delta) or carbamylated EPO (CEPO).

In another embodiment, the invention relates to a composition comprising a medicament known to have a tinnitus impairment side-effect in combination with a molecule having EPO activity selected from CENTO-528, CENTO-530, Dynepo and CEPO, said composition being for administration to a patient in need of such treatment.

### MODES FOR CARRYING OUT THE INVENTION

The invention embraces compositions and methods for prophylactic and therapeutic treatment of hearing impairments, particularly for the treatment of ototoxin-induced hearing impairments involving neuronal damage, loss or degeneration of neurons in a patient, or for the prevention of toxic side effects of ototoxic medications, by administration of one or more molecules having EPO activity such as EPO, a biosimilar, a variant, or a mutant thereof; a protein or peptide mimetic of EPO; or a small molecule mimetic of EPO.

Additionally, the invention also addresses compositions and methods for prophylactic and therapeutic treatment of balance impairments, particularly for the treatment of ototoxin-induced balance impairments involving neuronal damage, loss or degeneration of neurons in a patient, or for the prevention of toxic side effects of ototoxic medications, by administration of one or more molecules having EPO activity such as EPO, a biosimilar, a variant, or a mutant thereof; a protein or peptide mimetic of EPO; or a small molecule mimetic of EPO.

By “subject,” “individual,” or “patient” is meant an individual organism, preferably a vertebrate, more preferably a mammal, including humans, domestic, and farm animals, and zoo, sports, or pet animals, such as dogs, horses, cats, sheep, pigs, cows, etc. The preferred mammal herein is a human. The methods of the present invention are thus applicable to both human therapy and veterinary applications.

“Treating” a disease with the compounds and methods discussed herein is defined as administering one or more of the compounds discussed herein, with or without additional therapeutic agents, in order to reduce, eliminate, or reverse either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total. The compounds and methods of the invention can be administered to asymptomatic patients at risk of developing the clinical symptoms of the disease, in order to suppress the appearance of any adverse symptoms.

Such treatment is expected to allow hair cells and/or auditory neurons to tolerate intermittent insults from either environmental noise trauma or treatment with ototoxins, and to slow down, prevent or reverse the progressive degeneration of the auditory neurons and hair cells which is responsible for hearing loss in pathological conditions such as presbycusis (age-related hearing loss), inherited sensorineural degeneration, and post-idiopathic hearing losses, and to preserve the functional integrity of the inner ear. Such treatment will also support the auditory neurons for better and longer performance of cochlear implants.

“Treatment” refers to therapeutic treatment. “Prophylaxis” or “prevention” refers to prophylactic or preventative measures, wherein the object is to prevent or slow down (lessen) neuron-damage-related hearing impairment, preferably ototoxin-induced or inducible. A “prophylactically effective amount” is an amount sufficient to have a prophylactic or preventative effect.

Those in need of treatment include those already experiencing a hearing impairment, those prone to having the impairment, and those in which the impairments are to be prevented. The hearing impairments are due to neuronal damage, wherein the damage is caused by infections, mechanical injury, loud sounds, aging, or chemical-induced ototoxicity, wherein ototoxins include therapeutic drugs including antibiotics, antimicrobials, antifungals, anti-neoplastic agents, salicylates, quinines, contaminants in foods or medicaments, and environmental or industrial pollutants. Typically, treatment is performed to prevent or reduce ototoxicity, especially resulting from or expected to result from administration of therapeutic drugs. The treatment may be performed with a therapeutically effective composition given immediately after the exposure to prevent or reduce the ototoxic effect, or prior to or concomitantly with the ototoxic pharmaceutical or the exposure to the ototoxin.

“Balance impairment” refers to a neurologic disorder, oto-neurological, in which the patient displays, complains of, or is diagnosed to have known diagnostic symptoms of a balance disorder, including ataxic gait, inability to stand on one leg, or inability to walk heel-to-toe, inability to tandem walk, and dizziness or vertigo that are neurologically related. During vertigo the patient may experience a subjective impression of movement in space (subjective vertigo) or of objects moving in space (objective vertigo) usually with a loss of equilibrium. These impairments of interest to the present invention are those typically associated with damage to neurons, and possibly hair cells, of the vestibular system related to the eighth cranial nerve. Particularly affected may be neurons of the vestibule, semicircular canal, eighth nerve, vestibular neurons of the brainstem and their temporal lobe connections, and more particularly the organ of Corti.

“Ototoxic agent” refers to a substance that through its chemical action injures, impairs, or inhibits the activity of a component of the nervous system related to hearing or balance, which in turn impairs hearing or balance. A list of ototoxic agents that cause hearing or balance impairments is provided by the League for the Hard of Hearing; see URL World-Wide-Web.lhh.org/about_hearing_loss/understanding/OtotoxicBrochure.pdf, incorporated herein by reference in its entirety. This includes, but is not limited to, neoplastic agents such as vincristine, vinblastine, cisplatin, taxol, methotrexate, carboplatinum, bleomycin, nitrogen mustard; bromocryptine or dideoxy-compounds, e.g., dideoxyinosine; alcohol; metals; industrial toxins involved in occupational or environmental exposure, including toluene, xylene, etc; contaminants of food or medicaments; vitamins or therapeutic drugs, e.g., antibiotics such as penicillin, aminoglycosides (as described below), polypeptide antibiotics, minocycline, sulfonamides, vancomycin, amphotericin, or chloramphenicol; large doses of vitamins A, D, or B6; salicylates including aspirin and aspirin containing products, and salicylates and methylsalicylates; non-steroidal anti-inflammatory drugs (NSAIDS) including diclofenac, etodolac, ketorolac, fenprofen, ibuprofen, ketoprofen, indomethacin, naproxen, phenylbutazone, piroxicam, proglumetacin, proquazon, rofecoxib, tolmetin, xomepirac, and sulindac; narcotic analgesics including hydrocodone; mucosal protectants, including misoprostol; quinines and synthetic quinine-like compounds including chloroquine phosphate, quinacrine hydrochloride, and quinine sulfate; and loop diuretics including furosemide, ethacrynic acid, chlor-thalidone, bumetanide, acetazolamide, diapamide, hydrochlorothiazide, methylchlorothiazide, and bendroflumethiazide. Some additional ototoxic agents that are known to cause tinnitus includes vapors of solvents and other chemicals, including, cyclohexane, dichloromethane, hexane, lindane, methylchloride, methyl-n-butyl ketone, perchloroethylene, styrene, tetrachloroethane, toluene, trichloroethylene; cardiac medications including celiprolol, flecainide, lidocaine, metoprolol, procainamide, propranolol, and quinidine; psychopharmacologic agents, including amitryptiline, benzodiazepines such as alprazolam, clorazepate, chlordiazepoxide, diazepam, flurazepam, lorazepam, midazolam, oxazepam, prozepam, quazepam, temazepam, and triazolam, buproprion, carbamazepine, diclofensine, doxepin, desiprimine, fluoxetine, imipramine, lithium, melitracen, molindon, paroxetin, phenelzine, protriptilin, trazodon and zimeldin; glucocorticosteroids including prednisolone and adrenocorticotrophic hormone; anesthetics including bupivacain, tetracain and lidocaine; and thalidomide. By “exposure to an ototoxic agent” is meant that the ototoxic agent is made available to, or comes into contact with, a mammal. Exposure to an ototoxic agent can occur by direct administration, e.g., by ingestion or administration of a food, medicament, or therapeutic agent, e.g., a chemotherapeutic agent, by accidental contamination, or by environmental exposure, e.g., aerial or aqueous exposure.

“Aminoglycoside antibiotic” refers to a broad class of amino sugar containing antibiotics well known in the art. The aminoglycoside agents described in the literature which are useful in the methods of the present invention include, but are not limited to, amikacin (BB-K8), butirosin, geneticin, gentamicin, kanamycin, lividomycin, neomycin, paromomycin, hybrimycin, propikacin (UK 31214), ribostamycin, seldomycin, trehalosamine, α-D-mannosyl-α-D-glucosaminide, apramycin, bluensomycin, netromycin, streptomycin, sisomicin, destomycin, antibiotic A-396-I, dibekacin, kasugamycin, fortimicin, netilmicin, hygromycin, minocycline, capreomycin, amphotericin and tobramycin, and derivatives, analogs or variants thereof. Also useful in the methods of the invention are ototoxic glycopeptide antibiotics such as vancomycin, and ototoxic macrolide antibiotics such as erythromycin. In one embodiment, in any of the compositions and methods described herein where aminoglycoside antibiotics are used, gentamicin can be excluded.

“Platinum-containing antineoplastic agents” refers to a broad class of water-soluble, platinum coordination compounds well known in the art, typically having anti-tumor activity. The platinum-containing antineoplastic agents described in the literature which are useful in the methods of the present invention include, but are not limited to, cis-diaminedichloro-platinum(II) (cisplatin), trans-diaminedichloro-platinum(II), cis-diamine-diaquaplatinum(II)-ion, cis-diaminedichloroplatinum(II)-ion, chloro(diethylenetriamine)-platinum(II) chloride, dichloro(ethylenediamine)-platinum(II), diamine(1,1-cyclobutanedicarboxylato)-platinum(II) (carboplatin), spiroplatin, dichlorotrans-dihydroxybisisopropolamine platinum IV (iproplatin), diamine(2-ethylmalonato)platinum(II), ethylenediamine-malonatoplatinum(II), aqua(1,2-diaminodiclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), and (1,2-diaminocyclohexane)-oxalatoplatinum(II).

A “therapeutically effective amount” of a compound is an amount of the compound, which, when administered to a subject, is sufficient to reduce or eliminate either a disease or one or more symptoms of a disease, or to retard or reverse the progression of a disease or of one or more symptoms of a disease, or to reduce the severity of a disease or of one or more symptoms of a disease, or to suppress the clinical manifestation of a disease, or to suppress the manifestation of adverse symptoms of a disease.

An effective amount of a molecule having EPO activity to be employed therapeutically will depend, for example, upon the therapeutic objectives, the route of administration, the species of the patient, and the condition of the patient. Accordingly, it will be necessary for the therapist to titer the dosage and modify the route of administration as required to obtain the optimal therapeutic effect. As is known in the art, adjustments for age as well as the body weight, general health, sex, diet, time of administration, drug interaction and the severity of the disease may be necessary, and will be ascertainable with routine experimentation by those skilled in the art. A typical daily dosage of EPO used alone might range from about 1 μg/kg to up to 100 mg/kg of patient body weight or more per day, depending on the factors mentioned above, preferably about 10 μg/kg/day to 10 mg/kg/day. Typically, the clinician will administer EPO until a dosage is reached that repairs, maintains, and, optimally, reestablishes neuron function to relieve the hearing impairment. Generally, the molecule having EPO activity is formulated and delivered to the target site at a dosage capable of establishing at the site a level greater than about 0.1 ng/ml, more typically from about 0.1 ng/ml to 5 mg/ml, preferably from about 1 ng/ml to 2000 ng/ml, of the molecule having EPO activity.

If several molecules having EPO activity are administered together, they need not be administered by the same route or in the same formulation. However, they can be combined into one formulation as desired.

The molecule(s) having EPO activity optionally may be combined with or administered in concert with ototoxic pharmaceutical drugs. Initially the drugs are administered in conventional therapies known for the ototoxic pharmaceutical. Adjustments to the therapies are at the discretion of the skilled therapist to titrate dosages and conditions that decrease ototoxicity-related hearing while maintaining, and preferably improving, treatment outcomes with the ototoxic pharmaceutical drug.

Some pharmaceutical compositions comprise an effective ototoxicity-inhibiting amount of a molecule having EPO activity as described herein, a therapeutically effective amount of the ototoxic pharmaceutical drug, such as an aminoglycoside antibiotic, or an anti-neoplastic agent such as cisplatin, and optionally a pharmaceutically acceptable carrier and/or vehicle which would be familiar to one skilled in the pharmaceutical arts. The actual amounts of ototoxic pharmaceutical drug employed will range from those given in standard references for prescription drugs, e.g. “Physicians Desk Reference” (1995), “Drug Evaluations” AMA, 6th Edition (1986); to amounts somewhat larger since the ototoxicity potential is reduced in these compositions.

The effective amounts of such agents, if employed, will be at the physician's or veterinarian's discretion. Dosage administration and adjustment is done to achieve the best management of hearing or balance (and when used in conjunction with an ototoxic pharmaceutical drug, the indication for the ototoxic drug). The dose will additionally depend on such factors as the type of drug used and the specific patient being treated. Typically the amount employed will be the same dose as that used if the drug were to be administered without the molecule having EPO activity; however, higher doses may be employed depending on such factors as the presence of side-effects, the condition being treated, the type of patient, the type of molecule having EPO activity, and the type of ototoxic drug, provided the total amount of agents provides an effective dose for the condition being treated. For example, a test dose may be 5 mg, which is then ramped up to 10-20 mg per day, once a day, to 25 mg twice per day (BID) or three times per day (TID), and may be titrated to 50 mg BID or TID as the patient tolerates it. Tolerance level is estimated by determining whether decrease in hearing impairment is accompanied by signs of observed side-effects. A discussion of the dosage, administration, indications and contraindications associated with ototoxic pharmaceuticals optionally used with EPO in the methods of the invention can be found in the Physicians Desk Reference, Medical Economics Data Production Co., Montvale, N.J. (2000).

The effectiveness of treating hearing impairments with the methods of the invention can be evaluated by the following signs of recovery, including recovery of normal hearing function or balance function, which can be assessed by known diagnostic techniques including those discussed herein, and normalization of nerve conduction velocity, which is assessed electro-physiologically.

By a composition (or molecule, etc.) having “erythropoietin activity” is meant any composition (or molecule, etc.) having the full range of biological activity of human erythropoietin or at least one of the biological activities of EPO, such as the in vivo or in vitro activity of causing an increase in production of reticulocytes and/or red blood cells by bone marrow cells. Thus, molecules that lack the in vivo or in vitro activity of causing an increase in production of reticulocytes and/or red blood cells by bone marrow cells, but retain other biological activities of EPO, are also embraced by compositions or molecules having EPO activity.

Erythropoietin (EPO) has been the focus of significant research activity due to its utility in treating several serious diseases. EPO is currently approved in the United States for treatment of anemia in patients with chronic renal failure undergoing dialysis (recombinant human erythropoietin is sold under the brand name Epogen®, a registered trademark of Amgen, Inc., Thousand Oaks, Calif.). EPO is also believed to be useful in treatment of various other disorders; see, e.g., International Patent Publication No. WO 06/006165, directed to using EPO for enhancing immune responses and for the treatment of certain lympho-proliferative disorders; US 2006/0094648, directed to therapeutic or prophylactic treatment of myocardial ischemia, such as due to myocardial infarction, by administering erythropoietin; or US 2005/0272634, directed to using EPO for treatment of various disorders such as hypercholesterolemia, atherosclerosis, and diabetes.

Molecules having erythropoietin (EPO) activity include polypeptides and proteins having at least one of the biological activities of human erythropoietin. Molecules having erythropoietin activity include, but are not limited to, erythropoietin itself, recombinant human erythropoietin, erythropoietin analogs, erythropoietin biogenerics, erythropoietin biosimilars, erythropoietin isoforms, erythropoietin mimetics, erythropoietin fragments, hybrid erythropoietin proteins, mutants of any of the foregoing molecules, erythropoietins with covalent substitutions, and any of the foregoing molecules with variant glycosylation patterns, regardless of the biological activity of the same and further regardless of the method of synthesis or manufacture thereof, including but not limited to, recombinant, whether produced from cDNA or genomic DNA, synthetic, transgenic and gene activated methods. Some examples of commercially available preparations of erythropoietin include PROCRIT® (Epoetin alfa) (PROCRIT is a registered trademark of Johnson & Johnson Corp., New Brunswick, N.J., USA for an agent for the treatment of anemia), RETACRIT™ (Epoetin zeta) (RETACRIT is a registered trademark of Hospira, Inc., Lake Forest, Ill., USA for a drug for the treatment of anemia), EPREX® (EPREX is a registered trademark of Johnson & Johnson Corp., New Brunswick, N.J., USA for a drug for the treatment of anemia), and ERYPRO™ (ERYPRO is a trademark of Biocon, Bangalore, India for erythropoietin). Other molecules with EPO activity are disclosed in EP 640619, WO 05/051327; WO 99/66054, WO 99/38890, U.S. Pat. No. 5,688,679, WO 99/11781, EP 1064951, WO 98/05363, U.S. Pat. No. 5,643,575, WO 99/05268, WO 95/05465, WO 94/12650; and WO 91/05867; the disclosures of the use of those molecules as described in the respective patent publications are hereby incorporated by reference herein. Specific examples of cell lines modified for expression of endogenous human erythropoietin are described in PCT publications WO 99/05268 and WO 94/12650.

Erythropoietin-mimetics are molecules capable of acting as EPO in binding to the EPO receptor wherein the mimetic can have little or no similarity to native EPO. EPO mimetics are well known to those skilled in the art. Two kinds of EPO-mimetics have been described: peptides and non-peptides. Specific examples of erythropoietin mimetics are described in U.S. Pat. No. 5,767,078 and U.S. Pat. No. 5,773,569. Additional EPO-mimetics, such as CNTO-528, and CNTO-530 have been produced using Centocor's technology Mimetibody™ and described, for example, in PCT publications WO 08/042800 and WO 07/115148, US patent U.S. Pat. No. 7,241,733 and US patent publication US 2006/0051844. CNTO-530 is a 58 kD antibody Fc domain fusion protein, that contains two EMP1 sequences as a pharmacophore. CNTO-530 has no sequence homology with EPO but acts as a novel erythropoietin receptor agonist.

Small molecule EPO mimetics were discovered by scientists from Scripps, Affymax and Johnson Pharmaceutical Research Institute screening a peptide phage library to search for novel sequences that bound to EPO-R. One product resulting from this research is a pegylated peptide with no sequence homology to EPO but with EPO-R specificity, marketed as Hematide™ (Hematide is a registered trademark of Affymax, Inc., Palo Alto, Calif., USA, for a pharmaceutical preparation for use in stimulating human blood cell production). Some of these agents are described in Bunn, F, Blood, (2007) Vol 109 No. 3, 808-873.

Long-acting forms of EPO are also contemplated and may be preferred in some embodiments of the present invention for administration as the second or third exposure in a dosing segment. As used herein, a “long-acting EPO” includes sustained-release compositions and formulations of EPO with increased circulating half-life, typically achieved through modification such as reducing immunogenicity and clearance rate, and EPO encapsulated in polymer microspheres. Examples of “long-acting EPO” include, but are not limited to, conjugates of erythropoietin with polyethylene glycol (PEG) disclosed in PCT publication WO 02/049673 (Burg et al.), PEG-modified EPO disclosed in PCT publication WO 02/32957 (Nakamura et al.), conjugates of glycoproteins having erythropoietic activity and having at least one oxidized carbohydrate moiety covalently linked to a non-antigenic polymer disclosed in PCT publication WO 94/28024 (Chyi et al.), and other PEG-EPO prepared using SCM-PEG, SPA-PEG and SBA-PEG. Carbamylated EPO (CEPO) as described for example in PCT publication WO 06/014466 is also contemplated as a molecule with EPO activity in this invention, and may be preferred in some embodiments thereof.

“Erythropoiesis stimulating agents” (ESA) are substances that upregulate genes for, and/or expression and/or activity of, proteins besides EPO that are important in erythropoiesis including EPO-R, transferrin, transferrin receptor, or ferroportin. Some of these agents are also molecules which can be orally administered. The most advanced development of an oral ESA is a group of compounds originating from Fibrogen, now in co-development with Astellas for certain territories, now including Europe. These compounds up-regulate endogenous EPO by inhibition of hypoxia induced factor prolyl hydroxylase-(HIF-PH). They include FG-2216, FG-4539, FG-4592 and FG-6513. Some of these compounds are disclosed in US Publications US 2006/0178317, US 2006/0178316 and US 2006/0183695 and PCT publications WO 03/049686, WO 05/011696, WO 06/133391 and WO 07/146438, incorporated herein in their entirety.

By “molecule capable of increasing the endogenous EPO or stimulating erythropoiesis” is meant molecules that regulate the EPO gene as well as the interaction of EPO with EPO-R. These molecules can be proteins or peptides, or small molecules. Rather than being agents that directly stimulate and produce erythropoiesis by combining with the erythropoietin receptor, they actually cause the production of endogenous erythropoietin. By producing the erythropoietin, the agents are able to sustain lower but more sustained concentration of EPO, and it is the endogenous erythropoietin which then produces the erythropoiesis.

By “variant” is meant a modified peptide that retains its binding properties wherein the modifications include, but are not limited to, conservative substitutions in which one or more amino acids are substituted for other amino acids; deletion or addition of amino acids that have minimal influence on the binding properties or secondary structure; conjugation of a linker; and post-translation modifications such as, for example, the addition of functional groups. Conservative amino acid substitution is an amino acid substituted by an alternative amino acid of similar charge density, hydrophilicity/hydrophobicity, size, and/or configuration (e.g. Val for Be). Means of making such modifications are well known in the art. Tissue protective peptides derived from or sharing consensus sequences with portions of Erythropoietin (EPO), that are not involved in the binding of the ligand to the receptor complex have been described in PCT publications WO 07/019545 and WO 04/096148.

By “biosimilars” is meant copies of existing biotechnological products. Biosimilars are manufactured without access to the originator's molecular clone and original cell bank, and by a different fermentation and purification process. Although biosimilars are not identical to an existing approved product, they have demonstrated “comparability” to said approved product. Biosimilars are also sometimes referred to as “Follow-on biologics.”

By “erythropoietin biosimilars” is meant copies of existing erythropoietin products. Shire's Epoetin delta (Dynepo™, a registered trademark of Hoechst GmbH, Frankfurt, Germany, for pharmaceutical preparations for the treatment of cardiovascular and blood disorders), which sits somewhere in between a branded and a biosimilar drug is also contemplated in some embodiments of the present invention. Dynepo is produced by gene activation technology in a “human cell line.”

Formulation and Administration of Erythropoietin and Molecules with EPO Activity

Numerous formulations of erythropoietin are known in the art, such as the commercially available PROCRIT® (Epoetin alfa), RETACRIT™ (Epoetin zeta), EPREX®, and ERYPRO®. A wide variety of other formulations are also available; see, e.g., U.S. Pat. No. 4,806,524; U.S. Pat. No. 4,992,419; U.S. Pat. No. 5,376,632; U.S. Pat. No. 5,661,125; U.S. Pat. No. 6,120,761; and U.S. Pat. No. 7,129,267. Administration of erythropoietin is also well-known in the art, as described in the foregoing documents. EPO and molecules with EPO activity can be administered to a subject via parenteral administration, including, but not limited to, intravenous, intramuscular, subcutaneous, intraperitoneal, intracerebral, intraventricular, intracerebroventricular, intrathecal, intracisternal, intraspinal and perispinal administration. EPO can also be delivered continuously or semi-continuously via pump devices. EPO can also be delivered as “long-acting EPO” including sustained-release compositions and formulations of EPO with increased circulating half-life, typically achieved through modification such as reducing immunogenicity and clearance rate, and EPO encapsulated in polymer microspheres. The route of administration can be selected by the health care professional in accordance with known principles. When a molecule with EPO activity is administered, the formulation, dosage, and route of administration are also determined by the health care professional in accordance with known principles.

### Tests for Diagnosing Hearing Impairment

Tests are known and available for diagnosing hearing impairments. Neuro-otological, neuro-ophthalmological, neurological examinations, and electro-oculography can be used. (Wennmo et al. Acta Otolaryngol (1982) 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantify hearing loss, measured in decibels. With this device the hearing for each ear is measured, typically from 125 to 8000 Hz, and plotted. The speech recognition threshold, the intensity at which speech is recognized as a meaningful symbol, can be determined at various speech frequencies. Speech or phoneme discrimination can also be determined and used as an indicator of sensorineural hearing loss since analysis of speech sounds relies upon the inner ear and the eighth nerve. Tympanometry can be used to diagnose conductive hearing loss and aid in the diagnosis of those patients with sensorineural hearing loss. Electrocochleography, measuring the cochlear microphonic response and action potential of the eighth nerve, and evoked response audiometry, measured evoked response from the brainstem and auditory cortex, to acoustic stimuli can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including neural hearing loss. In sensory hearing loss, the sensation of loudness in the affected ear increases more with each increment in intensity than it does in the normal ear. Sensitivity to small increments in intensity can be demonstrated by presenting a continuous tone of 20 dB above the hearing threshold and increasing the intensity by 1 dB briefly and intermittently. The percentage of small increments detected yields the “short increment sensitivity index” value. High values, 80 to 100%, are characteristic of sensory hearing loss, whereas a neural lesion patient and those with normal hearing cannot detect such small changes in intensity. Pathologic adaptation is demonstrated when a patient cannot continue to perceive a constant tone above threshold of hearing, also known as tone decay. A Bekesy automatic audiometer or equivalent can be used to determine these clinical and diagnostic signs; audiogram patterns of the Type II pattern, Type III pattern and Type IV pattern are indicative of preferred hearing losses suitable for the treatment methods of the invention. As hearing loss can often be accompanied by vestibular impairment, vestibular function can be tested, particularly when presented with a sensorineural hearing loss of unknown etiology.

When possible, diagnostics for hearing loss, such as audiometric tests, should be performed prior to exposure in order to obtain a patient's normal hearing baseline. Upon exposure, particularly to an ototoxic drug, audiometric tests should be performed twice a week and testing should be continued for a period after cessation of the ototoxic drug treatment, since hearing loss may not occur until several days after cessation. U.S. Pat. No. 5,546,956 provides methods for testing hearing that can be used to diagnose the patient and monitor treatment. U.S. Pat. No. 4,637,402 provides a method for quantitatively measuring a hearing defect that can be used to diagnose the patient and monitor treatment.

Another diagnostic test for hearing loss is provided by Athena Diagnostics Inc (Worcester, Mass. 01605). Their OtoDX™ Aminoglycoside Hypersensitivity Test (#327) diagnoses sensorineural, nonsyndromic hearing loss often associated with aminoglycoside antibiotic exposure.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method for prevention in or treatment of an individual having or prone to having a hearing impairment, said method comprising administering a therapeutically effective amount of a composition comprising one or more molecules having erythropoietin activity, selected from EPO; an EPO biosimilar; an EPO variant; an EPO mutant; a protein or peptide mimetic of EPO; a small molecule mimetic of EPO; and an erythropoiesis stimulating agent.

2. (canceled)

3. The method of claim 1, wherein the molecule having EPO activity is selected from the group consisting of an EPO-mimetic antibody fusion protein selected from CNTO-528 and CNTO-530, and a molecule having EPO activity selected from Dynepo (Epoetin delta) and carbamylated EPO (CEPO).

4. (canceled)

5. The method of claim 1, wherein the hearing impairment is a result of neuronal damage, noise or acoustic trauma, or aging.

6-8. (canceled)

9. The method of claim 1, wherein the hearing impairment is a result of damage caused by an ototoxic agent, and wherein said ototoxic agent is selected from the group consisting of an aminoglycoside antibiotic, a chemotherapeutic agent, a salicylate or salicylate-like compound, a non-steroidal anti-inflammatory drug, a diuretic, a narcotic analgesic, and a quinine, with the proviso that if the aminoglycoside antibiotic is gentamicin, the molecule having erythropoietin activity is not EPO.

10-11. (canceled)

12. The method of claim 9, wherein said ototoxic agent is an aminoglycoside antibiotic, selected from the group consisting of neomycin, amikacin, tobramycin, viomycin, sisomicin, netimicin, streptomycin, dibexacin, fortimicin, monocycline, erythromycin, capreomycin, and dihydrostreptomycin.

13. The method of claim 9, wherein said ototoxic agent is the aminoglycoside antibiotic gentamicin and the molecule having erythropoietin activity is selected from an EPO biosimilar; an EPO variant; an EPO mutant; a protein or peptide mimetic of EPO; a small molecule mimetic of EPO and an erythropoiesis stimulating agent.

14. (canceled)

15. The method of claim 13, wherein the molecule having erythropoietin activity is an EPO mimetic antibody fusion protein selected from CNTO-528 and CNTO-530.

16. (canceled)

17. The method of claim 9, wherein the ototoxic agent is an anti-neoplastic drug selected from cisplatin and a cisplatin-like compound.

18-21. (canceled)

22. The method of claim 9, wherein the ototoxic agent is a salicylate or an NSAID selected from aspirin, salicylate, diclofenac, etodolac, ketorolac, fenprofen, ibuprofen, indomethacin, naproxen, piroxicam and sulindac.

23-24. (canceled)

25. The method of claim 1, wherein the hearing impairment is tinnitus.

26. A method of reversing hearing loss or recovering or enhancing hearing function, said method comprising administering to the mammal a therapeutically effective amount of a molecule having erythropoietin activity selected from EPO, or a biosimilar, a variant, or a mutant thereof; a protein or peptide mimetic of EPO; a small molecule mimetic of EPO; and an erythropoiesis stimulating agent.

27. The method of claim 26, wherein the molecule having erythropoietin activity is an EPO-mimetic antibody fusion protein selected from CNTO-528 and CNTO-530, or a molecule having EPO activity selected from Dynepo (Epoetin delta) and carbamylated EPO (CEPO).

28-30. (canceled)

31. A therapeutic composition for treating or preventing a hearing impairment caused by an ototoxic agent in a mammal, comprising a therapeutic amount of a combination of the ototoxic agent and a molecule having erythropoietin activity wherein said molecule having erythropoietin activity is selected from EPO, or a biosimilar, a variant, or a mutant thereof; a protein or peptide mimetic of EPO; a small molecule mimetic of EPO; and an erythropoiesis stimulating agent, for administration to the mammal in need of such treatment, with the proviso that if the ototoxic agent is gentamicin, the molecule having erythropoietin activity is not EPO.

32-34. (canceled)

35. The therapeutic composition of claim 31, wherein the molecule with EPO activity is selected from CNTO-528 and CNTO-530, or a molecule with EPO activity selected from Dynepo (Epoetin Delta) and carbamylated EPO (CEPO).

36-48. (canceled)

49. A method for preventing or treating prevention in or treatment of a mammal having or prone to having an ototoxin induced balance impairment ototoxin-induced balance impairment, said method comprising administering to the mammal a therapeutically effective amount of a composition comprising one or more molecules having-erythropoietin activity, selected from EPO; an EPO biosimilar; an EPO variant; an EPO mutant; a protein or peptide mimetic of EPO; a small molecule mimetic of EPO; and an erythropoiesis stimulating agent.

50-92. (canceled)
